(19) United States
(12) Patent Application Publication
(10) Pub. No.: US 2004/0213862 A1
(43) Pub. Date: Oct. 28, 2004

(54) Methods and formulations for inhibiting naturally occurring phosphodiesterase

(76) Inventors: Chen Su, West Jordan, UT (US); Afa Kehaati Palu, Orem, UT (US); Bing-Nan Zhou, Pleasant Grove, UT (US); Claude Jarakae Jensen, Cedar Hills, UT (US); Stephen Story, Alpine, UT (US)

Correspondence Address: Kirton & McConkie, Attorney for Applicant, 1800 Eagle Gate Tower, 60 East South Temple, Salt Lake City, UT, 84111, US

(21) Appl. No.: 10/798,730

(22) Filed: Mar. 11, 2004

Related U.S. Application Data
(60) Provisional application No. 60/458,204, filed on Mar. 27, 2003.

(51) Int. Cl. A61K 35/78

(52) U.S. Cl. 424/769; 424/777

(57) ABSTRACT

Methods and formulations for using Tahitian Noni juice for inhibiting naturally occurring phosphodiesterases (PDEs). The present invention relates to Tahitian Noni juice as a PDE inhibitor to increase body energy and reduce allergy and asthma reactions.

Related Applications

This application claims priority to United States Provisional Application Serial No. 60/458,204, filed Mar. 27, 2003 and entitled, “Inhibiting Effects of Tahitian Noni Puree Juice and Tahitian Noni Juice on Phosphodieterase Enzymes (PDE1, PDE2, PDE3, PDE4, PDE5, PDE6)”

### BACKGROUND

1. Field of the Invention

The present invention relates to methods and formulations for using Tahitian Noni juice for inhibiting naturally occurring phosphodiesterases (PDEs). In other words, the present invention relates to Tahitian Noni juice as a PDE inhibitor.

2. Background of the Invention and Related Art

Phosphodiesterases, also known as PDEs, is a group of enzymes found in the human body. PDEs react with a chemical, cAMP, in the body.

cAMP is needed for normal cell function. There are benefits in inhibiting PDEs. These benefits include: helping asthma and allergy patients, and boosting body energy. An allergy is an excessive response to allergens in one or more parts of the body. Asthma, an airway disease, is due to the excessive response in the airway. An allergy begins when antigens enter the body tissues, these cells meet antigens, and IgE antibodies are released. The IgE antibodies bind to mask cells in tissues. This, combined with the secretion of histamine results in an allergy. Thus controlling the release of IgE will stop an allergy.

Research has shown that cAMP controls the release of IgE. Using an inhibiting PDE will increase the level of cAMP in the body. Thus a PDE inhibitor will help allergy and asthma patients.

PDE inhibitors can also help asthma patients during an attack. During an asthma attack, the muscles around the airway are constricted and inflamed. cAMP relaxes the muscles around the airways. Inhibiting PDEs increases the level of cAMP, helping to relax the muscles around the airways and helping to fight and treat an asthma attack.

A PDE inhibitor also boosts body energy. The body produces energy by using glucose. A body in need of energy produces glucose by converting glucagons. This conversion process includes both liver cells and fat cells that include cAMP. The PDE inhibitor boosts body energy by increasing the level of cAMP.

Thus one skilled the art will appreciate that PDE inhibitors are beneficial for the reasons explained above.

### SUMMARY AND OBJECTS OF THE INVENTION

The present invention relates to methods and formulations for using Tahitian Noni juice for inhibiting naturally occurring phosphodiesterases (PDEs). In other words, the present invention relates to Tahitian Noni juice as a PDE inhibitor.

Therefore, it is an object of some embodiments of the present invention to inhibit PDEs through use of Tahitian Noni puree juice and Tahitian Noni® Juice.

The present invention comprises Morinda citrifolia compositions, each of which include one or more extracts from the Morinda citrifolia L. plant. The Morinda citrifolia extracts preferably include Morinda citrifolia fruit juice which is preferably present in an amount capable of maximizing the inhibition of the PDE enzyme without causing negative side effects when the composition is administered to a mammal.

Methods of the present invention comprise the administration or the consumption of Morinda citrifolia extracts in amounts that inhibit the PDE enzymes in mammals. Methods of the present invention also include obtaining the Morinda citrifolia compositions and extracts including Morinda citrifolia fruit juice and concentrates thereof.

One object of some embodiments of the present invention is to reduce allergy and asthma reactions. Another object of some embodiments of the present invention is to increase the body energy of a mammal.

One object of some embodiments of the present invention is to increase the efficacy of other PDE inhibitors or allergy medicines or drugs. Another object of some embodiments of the present invention is to provide an over-the-counter composition inhibiting PDEs in mammals without requiring a prescription.

In accordance with the invention as embodied and broadly described herein, the present invention features various methods and formulations for inhibiting naturally occurring PDEs.

### DETAILED DESCRIPTION OF THE PREFERRED EMBODIMENTS

It will be readily understood that the compositions and formulations of the present invention, as generally described herein, could be designed in and could comprise a wide variety of different variations. Thus, the following more detailed description of the embodiments of the formulations and methods of the present invention is not intended to limit the scope of the invention, as claimed, but is merely representative of the presently preferred embodiments of the invention.

The present invention relates to methods and formulations for using Tahitian Noni juice for inhibiting naturally occurring phosphodiesterase (PDE). In other words, the present invention relates to Tahitian Noni juice as a PDEs inhibitor.

### General Discussion of Morinda Citrifolia and the Methods used to Produce Processed Morinda Citrifolia Products

The Indian Mulberry or Noni plant, known scientifically as Morinda citrifolia L. (Morinda citrifolia), is a shrub, or small or medium-sized tree 3 to 10 meters high that grows in tropical coastal regions around the world. The plant is native to Southeast Asia and has spread in early times to a vast area from India to eastern Polynesia. It grows randomly in the wild, and it has been cultivated in plantations and small individual growing plots. Morinda citrifolia has somewhat rounded branches and evergreen, opposite (or spuriously alternate), dark, glossy, wavy, prominently-veined leaves. The leaves are broadly elliptic to oblong, pointed at both ends, 10-30 cm in length and 5-15 cm wide.

Morinda citrifolia flowers are contained in a fleshy, globose, head-like cluster and are small, white, 3 to 5 lobed, tubular, fragrant, and about 1.25 cm long. The flowers develop into compound fruits composed of many small drupes fused into an ovoid, ellipsoid or roundish, lumpy body, 5-10 cm long, 5-7 cm thick, with waxy, white or greenish-white or yellowish, semi-translucent skin. The fruit contains “eyes” on its surface, similar to a potato. The fruit is juicy, bitter, dull-yellow or yellowish-white, and contains numerous red-brown, hard, oblong-triangular, winged, 2-celled stones, each containing about 4 seeds. At maturity, they are creamy-white and edible, but have an unpleasant taste and odor. When fully ripe, the fruit has a pronounced odor like rancid cheese.

Although the fruit has been eaten by several nationalities as food, one common use of the Indian mulberry plant was as a red and yellow dye source. However, Morinda citrifolia has also been discovered to contain health enhancing compounds and/or enzymes that, among other things, aids in easing inflammation, calming feelings of anxiety, supporting weight management, and promoting circulatory health in humans. Moreover, Morinda citrifolia is considered to be an adaptogenic herb, a herb which supports balanced body systems by responding to the body's need for stimulation or relaxation.

Because the Morinda citrifolia fruit is for all practical purposes inedible, the fruit must be processed in order to make it palatable for human consumption and included in the naturaceutical used to treat fungal activity within the body. Processed Morinda citrifolia fruit juice can be prepared by separating seeds and peels from the juice and pulp of a ripened Morinda citrifolia fruit; filtering the pulp from the juice; and packaging the juice. Alternatively, rather than packaging the juice, the juice can be immediately included as an ingredient in another food product, frozen or pasteurized. In some embodiments, the juice and pulp can be pureed into a homogenous blend to be mixed with other ingredients. Other process include freeze drying the fruit and juice. The fruit and juice can be reconstituted during production of the final juice product. Still other processes include air drying the fruit and juices, prior to being masticated.

The present invention contemplates the use of fruit juice and/or puree fruit juice extracted from the Morinda Citrifolia plant and further processed into a naturaceutical formulation. Fruit juice or puree juice concentrate is also contemplated. In one exemplary embodiment, namely in regards to the process for producing Morinda citrifolia juice, the fruit is either hand picked or picked by mechanical equipment. The fruit can be harvested when it is at least one inch (2-3 cm) and up to 12 inches (24-36 cm) in diameter. The fruit may have a color ranging from a dark green through a yellow-green up to a white color, and gradations of color in between. The fruit is thoroughly cleaned after harvesting and before any processing of the juice occurs.

The fruit is allowed to ripen or age from 0 to 14 days, with most fruit being held from 2 to 3 days. The fruit is ripened or aged by being placed on equipment so it does not contact the ground. It is preferably covered with a cloth or netting material during aging, but can be aged without being covered. When ready for further processing the fruit is light in color, from a light green, light yellow, white or translucent color. The fruit is inspected for spoilage or for excessively green color and hard firmness. Spoiled and hard green fruit is separated from the acceptable fruit.

The ripened and aged fruit is preferably placed in plastic lined containers for further processing and transport. The containers of aged fruit may be held from 0 to 30 days. Most fruit containers are held for 7 to 14 days before processing. The containers can optionally be stored under refrigerated conditions prior to further processing. The fruit is unpacked from the storage containers and is processed through a manual or mechanical separator. The seeds and peel are separated from the juice and pulp.

The juice and pulp from the fruit may be packaged into containers for storage and transport. Alternatively, the juice and pulp can be immediately processed into a finished juice product. The containers may be stored in refrigerated, frozen, or room temperature conditions.

The Morinda citrifolia juice and pulp are preferably blended in a homogenous blend, after which they may be mixed with other ingredients, such as flavorings, sweeteners, nutritional ingredients, botanicals, and colorings. The finished juice product is preferably heated and pasteurized at a minimum temperature of 181° F. (83° C.) or higher up to 212° F. (100° C.).

Another product manufactured is Morinda citrifolia puree and puree juice, in either concentrate or diluted form. Puree is essentially the pulp a separated from the seeds and is different than the fruit juice product described herein.

In one embodiment, the product is filled and sealed into a final container of plastic, glass, or another suitable material that can withstand the processing temperatures. The containers are maintained at the filling temperature or may be cooled rapidly and then placed in a shipping container. The shipping containers are preferably wrapped with a material and in a manner to maintain or control the temperature of the product in the final containers.

The juice and pulp may be further processed by separating the pulp from the juice through filtering equipment. The filtering equipment preferably consists of, but is not limited to, a centrifuge decanter, a screen filter with a size from 1 micron up to 2000 microns, more preferably less than 500 microns, a filter press, reverse osmosis filtration., and any other standard commercial filtration devices. The operating filter pressure preferably ranges from 0.1 psig up to about 1000 psig. The flow rate preferably ranges from 0.1 g.p.m. up to 1000 g.p.m., and more preferably between 5 and 50 g.p.m. The wet pulp is washed and filtered at least once and up to 10 times to remove any juice from the pulp. The wet pulp typically has a fiber content of 10 to 40 percent by weight. The wet pulp is preferably pasteurized at a temperature of 181° F. (83° C.) minimum and then packed in drums for further processing or made into a high fiber product.

Drying may further process the wet pulp. The methods of drying may include freeze-drying, drum drying, tray drying, sun drying, and spray drying. The dried Morinda citrifolia pulp may include a moisture content in the range from 0.1 to 15 percent by weight and more preferably from 5 to 10 percent by weight. The dried pulp preferably has a fiber content in the range from 0.1 to 30 percent by weight, and more preferably from 5 to 15 percent by weight.

The high fiber product may include wet or dry Morinda citrifolia pulp, supplemental fiber ingredients, water, sweeteners, flavoring agents, coloring agents, and/or nutritional ingredients. The supplemental fiber ingredients may include plant based fiber products, either commercially available or developed privately. Examples of some typical fiber products are guar gum, gum arabic, soybean fiber, oat fiber, pea fiber, fig fiber, citrus pulp sacs, hydroxymethylcellulose, cellulose, seaweed, food grade lumber or wood pulp, hemicellulose, etc. Other supplemental fiber ingredients may be derived from grains or grain products. The concentrations of these other fiber raw materials typically range from 0 up to 30 percent, by weight, and more preferably from 10 to 30 percent by weight.

Typical sweeteners may include, but are not limited to, natural sugars derived from corn, sugar beet, sugar cane, potato, tapioca, or other starch-containing sources that can be chemically or enzymatically converted to crystalline chunks, powders, and/or syrups. Also sweeteners can consist of artificial or high intensity sweeteners, some of which are aspartame, sucralose, stevia, saccharin, etc. The concentration of sweeteners may be between from 0 to 50 percent by weight, of the formula, and more preferably between about 1 and 5 percent by weight.

Typical flavors can include, but are not limited to, artificial and/or natural flavor or ingredients that contribute to palatability. The concentration of flavors may range, for example, from 0 up to 15 percent by weight, of the formula. Colors may include food grade artificial or natural coloring agents having a concentration ranging from 0 up to 10 percent by weight, of the formula.

Typical nutritional ingredients may include vitamins, minerals, trace elements, herbs, botanical extracts, bioactive chemicals and compounds at concentrations from 0 up to 10 percent by weight. Examples of vitamins one can add to the fiber composition include, but are not limited to, vitamins A, B1 through B12, C, D, E, Folic Acid, Pantothenic Acid, Biotin, etc. Examples of minerals and trace elements one can add to the fiber composition include, but are not limited to, calcium, chromium, copper, cobalt, boron, magnesium, iron, selenium, manganese, molybdenum, potassium, iodine, zinc, phosphorus, etc. Herbs and botanical extracts include, but are not limited to, alfalfa grass, bee pollen, chlorella powder, Dong Quai powder, Ecchinacea root, Gingko Biloba extract, Horsetail herb, Indian mulberry, Shitake mushroom, spirulina seaweed, grape seed extract, etc. Typical bioactive chemicals may include, but are not limited to, caffeine, ephedrine, L-carnitine, creatine, lycopene, etc.

The juice and pulp can be dried using a variety of methods. The juice and pulp mixture can be pasteurized or enzymatically treated prior to drying. The enzymatic process begins with heating the product to a temperature between 75° F. and 135° F. It is then treated with either a single enzyme or a combination of enzymes. These enzymes include, but are not limited to, amylase, lipase, protease, cellulase, bromelin, etc. The juice and pulp may also be dried with other ingredients, such as those described above in connection with the high fiber product. The typical nutritional profile of the dried juice and pulp is 1 to 20 percent moisture, 0.1 to 15 percent protein, 0.1 to 20 percent fiber, and the vitamin and mineral content.

The filtered juice and the water from washing the wet pulp are preferably mixed together. The filtered juice may be vacuum evaporated to a brix of 40 to 70 and a moisture of 0.1 to 80 percent, more preferably from 25 to 75 percent. The resulting concentrated Morinda citrifolia L. juice may or may not be pasteurized. For example, the juice would not be pasteurized in circumstances where the sugar content or water activity was sufficiently low enough to prevent microbial growth. It is packaged for storage, transport and/or further processing.

The Indian Mulberry plant or Morinda citrifolia is rich in natural ingredients. Those ingredients that have been discovered include: from the leaves—alaninc, anthraquinones, arginine, ascorbic acid, aspartic acid, calcium, beta-carotene, cysteine, cystine, glycine, glutamic acid, glycosides, histidine, iron, leucine, isoleucine, methionine, niacin, phenylalanine, phosphorus, proline, resins, riboflavin, serine, beta-sitosterol, thiamine, threonine, tryptophan, tyrosine, ursolic acid, and valine; from the flowers—acacetin-7-o-beta-d(+)-glucopyranoside, 5,7-dimethyl-apigenin-4′-o-beta-d(+)-galactopyranoside, and 6,8-dimethoxy-3-methylanthraquinone-1-o-beta-rhamnosyl-glucopyranoside; (from the fruit) acetic acid, asperuloside, butanoic acid, benzoic acid, benzyl alcohol, 1-butanol, caprylic acid, decanoic acid, (E)-6-dodeceno-gamma-lactone, (Z,Z,Z)-8,1 1,14-eicosatrienoic acid, elaidic acid, ethyl decanoate, ethyl hexanoate, ethyl octanoate, ethyl palmitate, (Z)-6-(ethylthiomethyl) benzene, eugenol, glucose, heptanoic acid, 2-heptanone, hexanal, hexanamide, hexanedioic acid, hexanoic acid (hexoic acid), 1-hexanol, 3-hydroxy-2-butanone, lauric acid, limonene, linoleic acid, 2-methylbutanoic acid, 3-methyl-2-buten-1-ol, 3-methyl-3-buten-1-ol, methyl decanoate, methyl elaidate, methyl hexanoate, methyl 3-methylthio-propanoate, methyl octanoate, methyl oleate, methyl palmitate, 2-methylpropanoic acid, 3-methylthiopropanoic acid, myristic acid, nonanoic acid, octanoic acid (octoic acid), oleic acid, palmitic acid, potassium, scopoletin, undecanoic acid, (Z,Z)-2,5-undecadien-1-ol, and vomifol; from the roots—anthraquinones, asperuloside (rubichloric acid), damnacanthal, glycosides, morindadiol, morindine, morindone, mucilaginous matter, nor-damnacanthal, rubiadin, rubiadin monomethyl ether, resins, soranjidiol, sterols, and trihydroxymethyl anthraquinone-monomethyl ether; from the root bark—alizarin, chlororubin, glycosides (pentose, hexose), morindadiol, morindanigrine, morindine, morindone, resinous matter, rubiadin monomethyl ether, and soranjidiol; from the wood—anthragallol-2,3-dimethylether; and from the tissue culture—damnacanthal, lucidin, lucidin-3-primeveroside, and morindone-6beta-primeveroside; from the plant—alizarin, alizarin-alpha-methyl ether, anthraquinones, asperuloside, hexanoic acid, morindadiol, morindone, morindogenin, octanoic acid, and ursolic acid.

Recently, as mentioned, many health benefits have been discovered stemming from the use of products containing Morinda citrifolia. One benefit of Morinda citrifolia is found in its ability to isolate and produce Xeronine, which is a relatively small alkaloid physiologically active within the body. Xeronine occurs in practically all healthy cells of plants, animals and microorganisms. Even though Morinda citrifolia has a negligible amount of free Xeronine, it contains appreciable amounts of the precursor of Xeronine, called Proxeronine. Further, Morinda citrifolia contains the inactive form of the enzyme Proxeronase which releases Xeronine from Proxeronine. A paper entitled, “The Pharmacologically Active Ingredient of Noni” by R. M. Heinicke of the University of Hawaii, indicates that Morinda citrifolia is “the best raw material to use for the isolation of xeronine,” because of the building blocks of Proxeronine and Proxeronase. These building blocks aid in the isolation and production of Xeronine within the body. The function of the essential nutrient Xeronine is fourfold.

First, Xeronine serves to activate dormant enzymes found in the small intestines. These enzymes are critical to efficient digestion, calm nerves, and overall physical and emotional energy.

Second, Xeronine protects and keeps the shape and suppleness of protein molecules so that they may be able to pass through the cell walls and be used to form healthy tissue. Without these nutrients going into the cell, the cell cannot perform its job efficiently. Without Proxeronine to produce Xeronine our cells, and subsequently the body, suffer.

Third, Xeronine assists in enlarging the membrane pores of the cells. This enlargement allows for larger chains of peptides (amino acids or proteins) to be admitted into the cell. If these chains are not used they become waste.

Fourth, Xeronine, which is made from Proxeronine, assists in enlarging the pores to allow better absorption of nutrients.

Each tissue has cells which contain proteins which have receptor sites for the absorption of Xeronine. Certain of these proteins are the inert forms of enzymes which require absorbed Xeronine to become active. Thus Xeronine, by converting the body's procollagenase system into a specific protease, quickly and safely removes the dead tissue from skin. Other proteins become potential receptor sites for hormones after they react with Xeronine. Thus the action of Morinda citrifolia in making a person feel well is probably caused by Xeronine converting certain brain receptor proteins into active sites for the absorption of the endorphin, the well being hormones. Other proteins form pores through membranes in the intestines, the blood vessels and other body organs. Absorbing Xeronine on these proteins changes the shape of the pores and thus affects the passage of molecules through the membranes.

Because of its many benefits, Morinda citrifolia has been known to provide a number of anecdotal effects in individuals having cancer, arthritis, headaches, indigestion, malignancies, broken bones, high blood pressure, diabetes, pain, infection, asthma, toothaches, blemishes, immune system failure, and others.

The compositions containing Morinda citrifolia may be in a form suitable for oral use, systemic administration, injection, and others. In regards to an oral composition, such a composition may exist, for example, as tablets, or lozenges, aqueous or oily suspensions, dispersible powders or granules, emulsions, syrups or elixirs. Compositions intended for oral use may be prepared according to any method known in the art for the manufacture of Morinda citrifolia compositions and such compositions may contain one or more agents selected from the group consisting of sweetening agents, flavoring agents, coloring agents and preserving agents. Tablets contain Morinda citrifolia in admixture with non-toxic pharmaceutically acceptable excipients which are suitable for the manufacture of tablets. These excipients may be for example, inert diluents, granulating and disintegrating agents, binding agents, and lubricating agents. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Aqueous suspensions contain the Morinda citrifolia in admixture with excipients suitable for the manufacture of aqueous suspensions. Such excipients are suspending agents, for example, sodium carboxymethyl-cellulose, methylcellulose, hydroxy-propylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitor monooleate, or condensation products of ethylene oxide with partial esters derived from fatty acids and hexitol anhydrides, for example polyethylene sorbitan monooleate.

Favorably, this invention provides a method inhibiting PDEs with a Morinda citrifolia-based naturaceutical formulation without any significant tendency to cause gastric side effects.

As used herein, the term Morinda citrifolia juice refers to a product that includes juice processed from the fruit of the Indian Mulberry or Morinda citrifolia L. plant. In one embodiment, Morinda citrifolia juice includes reconstituted fruit juice from pure juice puree of French Polynesia. The naturaceutical composition or formulation comprising at least one processed Morinda citrifolia product may also include other natural juices, such as a natural grape juice concentrate, a natural blueberry juice concentrate, and/or another natural juice concentrates. In a further embodiment, Morinda citrifolia juice is not processed from dried or powdered Morinda citrifolia.

### Morinda Citrifolia Based Naturaceutical Formulations and Methods of Administration for Inhibiting Naturally Occurring PDE within the Body

The present invention advances PDE inhibitors by providing a naturaceutical composition formulated with one or more processed Morinda citrifolia products derived from the Indian Mulberry plant. The Morinda citrifolia is incorporated into various carriers or naturaceutical compositions suitable for in vivo treatment of a patient. For instance, the inhibitor may be ingested, injected, introduced intravenously, or otherwise internalized as is appropriate and directed.

In one embodiment, the administration of Morinda citrifolia inhibits the action of a PDE enzyme, thereby increasing the level of cAMP. The increase in the level of cAMP causes an increase in IgE, which in turn, decreases the secretion of histamine. The decrease in histamine decreases the allergic reaction.

In another embodiment, the administration of Morinda citrifolia inhibits the action of a PDE enzyme, thereby increasing the level of cAMP. The increase in the level of cAMP causes the muscle around a mammal's airways to relax, which decreases the severity of the reaction to asthma.

In another embodiment, the administration of Morinda citrifolia inhibits the action of a PDE enzyme, thereby increasing the level of cAMP. The increase in the level of cAMP increases the amount of glucose produced by a mammal, increasing the body energy of the mammal.

In one exemplary embodiment, the naturaceutical composition of the present invention comprises one or more of a processed Morinda citrifolia (e.g. Morinda citrifolia fruit juice or fruit juice or puree juice) product present in an amount by weight between about 0.01 and 100 percent by weight, and preferably between 0.01 and 95 percent by weight. Several embodiments of formulations are provided below. However, these are only intended to be exemplary as one ordinarily skilled in the art will recognize other formulations or compositions comprising the processed Morinda citrifolia product.

The processed Morinda citrifolia product may be formulated with various other ingredients to produce various compositions, such as a naturaceutical composition, a topical dermal composition, or others. The ingredients to be utilized in a naturaceutical composition are any that are safe for introduction into the body of a mammal, and particularly a human, and may exist in various forms, such as liquids, tablets, lozenges, aqueous or oily solutions, dispersible powders or granules, emulsions, syrups, elixirs, etc. Moreover, since the naturaceutical composition is preferably consumed orally, it may contain one or more agents selected from the group consisting of sweetening agents, flavoring agents, coloring agents, preserving agents, and other medicinal agents as directed.

The ingredients to be utilized in a topical dermal composition are also any that are safe for internalizing into the body of a mammal and may exist in various forms, such as gels, lotions, creams, ointments, etc., each comprising one or more carrier agents. The ingredients for systemically (e.g. intravenously) administered formulations may also comprise any known in the art.

The present invention further features a method of administering a naturaceutical composition to a mammal to inhibit PDE within the body. In one exemplary embodiment, the method comprises the steps of (a) formulating a naturaceutical composition comprising in part a processed Morinda citrifolia product present in an amount between about 0.01 and 95 percent by weight, wherein the composition also comprises a carrier, such as water or purified water, and may also comprise other natural or artificial ingredients; (b) administering the naturaceutical composition into the body of a mammal, such that the processed Morinda citrifolia product is sufficiently internalized; (c) repeating the above steps as often as necessary to provide an effective amount of the processed Morinda citrifolia product needed to inhibit PDEs.

The step of administering the naturaceutical composition into the body preferably comprises ingesting the composition orally through one of several means. Specifically, the naturaceutical composition may be formulated as a liquid, gel, solid, or some other type that would allow the composition to be quickly digested and concentrated within the colon. It is important to note that the step of administering the naturaceutical composition should be carried out in an effective manner so that the greatest concentration of naturaceutical composition is allowed to absorb into the tissues and cells. For the naturaceutical composition to take effect, it must be sufficiently internalized. Once sufficiently internalized, it may then begin to effectuate the inhibition of PDEs.

In another embodiment, the step of administering the naturaceutical composition may include injecting the composition into the body using an intravenous pump. This technique is advantageous as it would allow the composition to be localized in the area where it would have the most effect, or the area that would provide for the greatest concentration of the naturaceutical composition.

In one exemplary embodiment, the naturaceutical composition is administered by taking between 1 teaspoon and 2 oz., and preferably 2 oz., of the naturaceutical composition every two hours each day, or at least twice a day on a continued basis. Also, the naturaceutical composition is to be taken on an empty stomach, meaning at a period of time at least two hours prior to consumption of any food or drink. Of course, one ordinarily skilled in the art will recognize that the amount of composition and frequency of use may vary from individual to individual.

The following tables illustrate or represent some of the preferred formulations or compositions contemplated by the present invention. As stated, these are only intended as exemplary embodiments and are not to be construed as limiting in any way.

| | |
|---|---|
| Ingredients | Percent by Weight |

Formulation One

| | |
|---|---|
| Morinda citrifolia puree juice or fruit juice | 100% |

Formulation Two

| | |
|---|---|
| Morinda citrifolia fruit juice | 85-99.99% |
| Water | 0.1-15% |

Formulation Three

| | |
|---|---|
| Morinda citrifolia fruit juice | 85-99.99% |
| non-Morinda citrifolia-based fruit juices | 0.1-15% |

Formulation Four

| | |
|---|---|
| Morinda citrifolia fruit juice | 50-90% |
| water | 0.1-50% |
| non-Morinda citrifolia-based fruit juices | 0.1-30% |

Formulation Five

| | |
|---|---|
| Morinda citrifolia puree juice | 85-99.9% |
| water | 0.1-15% |

Formulation Six

| | |
|---|---|
| Morinda citrifolia puree juice | 85-99.9% |
| non-Morinda citrifolia-based fruit juices | 0.1-15% |

Formulation Seven

| | |
|---|---|
| Morinda citrifolia puree juice | 50-90% |
| water | 0.1-50% |
| non-Morinda citrifolia-based fruit juices | 0.1-30% |

Formulation Eight

| | |
|---|---|
| Morinda citrifolia dietary fiber | 0.1-30% |
| water | 1-99.9% |
| non-Morinda citrifolia-based fruit juices | 1-99.9% |

Formulation Nine

| | |
|---|---|
| Morinda citrifolia dietary fiber | 0.1-30% |
| water | 1-99.9% |
| Morinda citrifolia fruit juice or puree juice | 1-99.9% |

Formulation Ten

| | |
|---|---|
| Morinda citrifolia oil | 0.1-30% |
| carrier medium | 70-99.9% |
| other ingredients | 1-95% |

Formulation Eleven

| | |
|---|---|
| Morinda citrifolia product | 10-80% |
| carrier medium | 20-90% |

Formulation Twelve

| | |
|---|---|
| Morinda citrifolia product | 5-80% |
| carrier medium | 20-95% |

Formulation Thirteen

| | |
|---|---|
| Morinda citrifolia oil or oil extract | 0.1-20% |
| carrier medium | 20-90% |

Formulation Fourteen

| | |
|---|---|
| Morinda citrifolia puree juice or fruit Juice | 0.1-80% |
| Morinda citrifolia oil | 0.1-20% |
| carrier medium | 20-90% |

Formulation Fifteen

| | |
|---|---|
| Morinda citrifolia puree juice concentrate | 100% |
| or fruit juice concentrate | |

Formulation Sixteen

| | |
|---|---|
| Morinda citrifolia fruit juice concentrate or | 85-99.99% |
| puree juice concentrate | |
| Water | 0.1-15% |

Formulation Seventeen

| | |
|---|---|
| Morinda citrifolia puree juice or fruit juice fraction | 100% |

Formulation Eighteen

| | |
|---|---|
| Morinda citrifolia fruit juice fraction | 85-99.99% |
| Water | 0.1-15% |

Formulation Nineteen

| | |
|---|---|
| Morinda citrifolia fruit juice fraction | 85-99.99% |
| non-Morinda citrifolia-based fruit juices | 0.1-15% |

Formulation Twenty

| | |
|---|---|
| Morinda citrifolia fruit juice fraction | 50-90% |
| water | 0.1-50% |
| non-Morinda citrifolia-based fruit juices | 0.1-30% |

Formulation Twenty One

| | |
|---|---|
| Morinda citrifolia puree juice fraction | 85-99.9% |
| water | 0.1-15% |

Formulation Twenty Two

| | |
|---|---|
| Morinda citrifolia puree juice fraction | 85-99.9% |
| non-Morinda citrifolia-based fruit juices | 0.1-15% |

As stated, in one exemplary embodiment, the present invention features a method for introducing an internal composition or formulation to inhibit PDEs. This method essentially comprises the introduction of a naturaceutical internal composition into the body of a mammal. Several embodiments of the internal composition comprising various different ingredients are contemplated for use, with each embodiment comprising one or more forms of a processed Morinda citrifolia product as taught and explained herein and a carrier agent or medium.

In one preferred method, PDEs are inhibited by administering at least one (1) ounce of one of Formulations One through Sixteen above in the morning on an empty stomach, and at least one (1) ounce at night on an empty stomach, just prior to retiring to bed.

In one example, which is not meant to be limiting in any way, the beneficial Morinda Citrifolia is processed into Tahitian Noni® juice manufactured by Morinda, Incorporated of Orem, Utah.

In an exemplary embodiment, the internal composition comprises the ingredients of: a processed Morinda citrifolia product present in an amount by weight between about 10-80 percent; and a carrier medium present in an amount by weight between about 20-90 percent.

In this embodiment, the processed Morinda citrifolia product may comprise one or more of a processed Morinda citrifolia fruit juice, processed Morinda citrifolia puree juice, processed Morinda citrifolia fruit or puree juice concentrate, processed Morinda citrifolia dietary fiber, and/or processed Morinda citrifolia oil extract product.

In another exemplary embodiment, the internal composition comprises the ingredients of: processed Morinda citrifolia fruit juice or puree juice present in an amount by weight between about 0.1-80 percent; processed Morinda citrifolia oil present in an amount by weight between about 0.1-20 percent; and a carrier medium present in an amount by weight between about 20-90 percent. Morinda citrifolia puree juice or fruit juice may also be formulated with a processed Morinda citrifolia dietary fiber product present in similar concentrations.

According to the present invention, the particular methods of introducing an internal composition may comprises any method of actually introducing the internal composition into the body of a mammal for the purposes identified herein. Although the particular methods are many, the present invention recognizes that the internal composition may be introduced intravenously, transdermally, orally, or systemically. No matter what method is employed, it is important to thoroughly internalize the composition so that the internal composition, and particularly the Morinda citrifolia and other active ingredients, can effectively inhibit or treat fungal and other microbial activity or growth.

The carrier medium identified in the above-identified Formulations may comprise any ingredient capable of being introduced into the body of a mammal, and that is also capable of providing the carrying medium to the processed Morinda citrifolia product. Specific carrier mediums formulations are well known in the art and not described in detail herein. The purpose of the carrier medium is as stated, to provide a means to embody the processed Morinda citrifolia product within the internal composition that is capable of being introduced into the body.

The following examples and research information set forth present how Tahitian Noni puree juice and Tahitian Noni® Juice successfully inhibits PDEs. These examples are not intended to be limiting in any way, but are merely illustrative of the benefits and advantages as well as the remedial effects of the Morinda citrifolia products.

### EXAMPLE1

The information in this report has two parts. First, is the inhibitory effect of Tahitian Noni Puree Juice Concentrate (TNPJCon) on the Phosphodiesterase enzyme (PDEs). Second, is the inhibitory effect of the Tahitian Noni International Brand Tahitian Noni® Juice on Phosphodiesterase which also includes the IC₅₀ determination.

1. Tahitian Noni Puree Juice Concentrate

The test material (TNPJCon) was tested using an Enzyme assays that involve PDE enzymes from both bovine and human origin known as PDE1, PDE2, PDE3, PDE4, PDE6. All of the enzyme assays were run on a 1% concentration of TNPJCon. Please note that the IC₅₀ was not determined.

Results

| 1% Concentration | |
|---|---|
| of TNPJCon | % Inhibition |
| PDE1 | 86 |
| PDE2 | 57 |
| PDE3 | 87 |
| PDE4 | 94 |
| PDE6 | 99 |

2. Tahitian Noni® Juice

Using the same protocol and enzymes used in the testing of the PDE inhibitory effects of TNPJCon indicated above, the inhibitory effects of Tahitian Noni® Juice was determined. Please note that the concentration changed from that used in the TNPJCon and again the IC₅₀ was not determined at this stage.

Results

| 2.1% Concentration | |
|---|---|
| of TNJ | % Inhibition |
| PDE1 | 67 |
| PDE2 | 72 |
| PDE3 | 86 |
| PDE4 | 99 |
| PDE6 | 96 |

The results from these enzyme bioassays were significant and compared very favorably with the reference compounds IBMX and Zaprinast. Therefore the IC₅₀ was determined from using the same experiments described above but in a range of concentrations from 0.1%, 0.3%, 1%, 3% and 10% concentration. The results are summary of two replicates for each concentration to a total of 10 per PDEs.

| Phosphodiesterase | TNJ Concentration | IC₅₀ |
|---|---|---|
| PDE1 | 3% | 3.25% |
| PDE2 | 10% | 3.52 |
| PDE3 | 1% | 0.83% |
| PDE4 | 1% | 0.69% |
| PDE5 | 3% | 2.35% |
| PDE6 | 0.3% | 0.33% |

From these data, we can rearrange them in their order of potency, meaning the order of how much Tahitian Noni® Juice is required to inhibit 50% of a certain PDE. More potent will be listed #1 and down to the least potent #6.

| Phosphodiesterase | IC₅₀ |
|---|---|
| 1. PDE6 | 0.33% |
| 2. PDE4 | .069% |
| 3. PDE3 | .083% |
| 4. PDE5 | 2.35% |
| 5. PDE1 | 3.25% |
| 6. PDE2 | 3.52% |

### EXAMPLE 2

### Summary

### Study Objection

To evaluate, in Enzyme assays, the activity of compound MDA-1 (PT# 1010069).

### Methods

Methods employed in this study have been adapted from the scientific literature to maximize reliability and reproducibility. Reference standards were run as an integral part of each assay to ensure the validity of the results obtained. Assays were performed under conditions described in the accompanying “Methods” section of this report. The literature reference(s) for each assay are in the “Literature References” section. If either of these sections were not originally requested with the accompanying report, please contact us at the number below for a printout of either of these report sections.

### Results

A summary of results meeting the significance criteria is presented in the following sections. Complete results are presented under the section labeled “Experimental Results”. Individual responses, if requested, are presented in the appendix to this report.

### Summary/Conclusion

Significant results are displayed in the following table(s) in rank order of potency for estimated IC₅₀ values.

### Summary of Significant Primary Results

Biochemical assay results are presented as the percent inhibition of specific binding or activity throughout the report. All other results are expressed in terms of that assay's quantitation method (see Methods section).

For primary assays, only the lowest concentration with a significant response judged by the assays' criteria, is shown in this summary.

Where applicable, either the secondary assay results with the lowest dose/concentration meeting the signjficance criteria or, if inactive, the highest dose/concentration that did not meet the significance criteria is shown.

Unless otherwise requested, primary screening in duplicate with quantitative data (e.g., IC50±SEM, Ki±SEM and nH) are shown where applicable for individual requested assays. In screening packages, primary screening in duplicate with semi-quantitative data (e.g., estimated IC50, Ki and nH) are shown where applicable (concentration range of 4 log units); available secondary functional assays are carried out (30 μM) and MEC or MIC determined only if active in primary assays >50% at 1 log unit below initial test concentration.

Please see Experimental Results section for details of all responses. Significant responses (250% inhibition or stimulation for Biochemical assays) were noted in the primary assays below:

Primary Tests

| Cat. # | Primary Biochemical Assay | Species | Conc. | % Inh. | IC₅₀* | Kᵢ | n_{H} |
|---|---|---|---|---|---|---|---|
| 146000 | Phosphodiesterase PDE 1 | bov | 3% | 58 | 3.25% | | |
| 148000 | Phosphodiesterase PDE 2 | hum | 10% | 87 | 3.52% | | |
| 152000 | Phosphodiesterase PDE 3 | hum | 1% | 54 | 0.83% | | |
| 154000 | Phosphodiesterase PDE 4 | hum | 1% | 64 | 0.69% | | |
| 156000 | Phosphodiesterase PDE 5 | hum | 3% | 59 | 2.35% | | |
| 156100 | Phosphodiesterase PDE 6 | bov | 0.3% | 55 | 0.33% | | |

Above Primary Tests in Rank Order of Potency

| | Primary | | | | | | |
|---|---|---|---|---|---|---|---|
| | Biochemical | | | | | | |
| Cat. # | Assay | Species | Conc. | % Inh. | IC₅₀* | Kᵢ | n_{H} |
| 156100 | Phosphodiesterase | bov | 0.3% | 55 | 0.33% | | |
| | PDE 6 | | | | | | |

*A standard error of the mean is presented where results are based on multiple, independent determinations.
bov = bovine;
hum = human

Above Primary Tests in Rank Order of Potency

| Cat. # | Primary Biochemical Assay | Species | Conc. | % Inh. | IC₅₀* | Kᵢ | n_{H} |
|---|---|---|---|---|---|---|---|
| 154000 | Phosphodiesterase PDE 4 | hum | 1% | 64 | 0.69% | | |
| 152000 | Phosphodiesterase PDE 3 | hum | 1% | 54 | 0.83% | | |
| 156000 | Phosphodiesterase PDE 5 | hum | 3% | 59 | 2.35% | | |
| 146000 | Phosphodiesterase PDE 1 | bov | 3% | 58 | 3.25% | | |
| 148000 | Phosphodiesterase PDE 2 | hum | 10% | 87 | 3.52% | | |

*A standard error of the mean is presented where results are based on multiple, independent determinations.
bov = bovine;
hum = human

Methods—Enzyme Assays

| | |
|---|---|
| 146000 Phosphodiesterase PDE1 | |
| Source: | Bovine heart |
| Substrate: | 1.01 μM (³H) cAMP + cAMP |
| Vehicle: | 1% H₂O |
| Pre-Incubation Time/Temp: | None |
| Incubation Time/Temp: | 20 minutes @ 25° C. |
| Incubation Buffer: | 50 mM Tris-HCl, 5 mM MgCl₂, 2 mM |
| | Cacl₂, 10 unit Calmodulin, pH |
| | 7.5 |
| Quantitation Method: | Quantitation of [³H]adenosine |
| Significance Criteria: | ≧50% of max stimulation or |
| | inhibition |
| 148000 Phosphodiesterase PDE2 | |
| Source: | Human platelets |
| Substrate: | 25.1 μM (³H) cAMP + cAMP |
| Vehicle: | 1% H₂O |
| Pre-Incubation Time/Temp: | None |
| Incubation Time/Temp: | 20 minutes @ 25° C. |
| Incubation Buffer: | 50 mM Tris-HCl, 5 mM MgCl₂, pH |
| | 7.5 |
| Quantitation Method: | Quantitation of [³H]adenosine |
| Significance Criteria: | ≧50% of max stimulation or |
| | inhibition |
| 152000 Phosphodiesterase PDE3 | |
| Source: | Human platelets |
| Substrate: | 1.01 μM (³H) cAMP + cAMP |
| Vehicle: | 1% H₂O |
| Pre-Incubation Time/Temp: | None |
| Incubation Time/Temp: | 20 minutes @ 25° C. |
| Incubation Buffer: | 50 mM Tris-HCl, 5 mM MgCl₂, pH |
| | 7.5 |
| Quantitation Method: | Quantitation of [³H]adenosine |
| Significance Criteria: | ≧50% of max stimulation or |
| | inhibition |
| 154000 Phosphodiesterase PDE4 | |
| Source: | Human U937 cells |
| Substrate: | 1.01 μM (³H) cAMP + cAMP |
| Vehicle: | 1% H₂O |
| Pre-Incubation Time/Temp: | None |
| Incubation Time/Temp: | 20 minutes @ 25° C. |
| Incubation Buffer: | 50 mM Tris-HCl, 5 mM MgCl₂, pH |
| | 7.5 |
| Quantitation Method: | Quantitation of [³H]adenosine |
| Significance Criteria: | ≧50% of max stimulation or |
| | inhibition |
| 156000 Phosphodiesterase PDE5 | |
| Source: | Human platelets |
| Substrate: | 1.01 μM (³H) cGMP + cGMP |
| Vehicle: | 1% H₂O |
| Pre-Incubation Time/Temp: | None |
| Incubation Time/Temp: | 20 minutes @ 25° C. |
| Incubation Buffer: | 50 mM Tris-HCl, 5 mM MgCl₂, pH |
| | 7.5 |
| Quantitation Method: | Quantitation of [³H]adenosine |
| Significance Criteria: | ≧50% of max stimulation or |
| | inhibition |
| 156100 Phosphodiesterase PDE6 | |
| Source: | Bovine retinal rod out segments |
| Substrate: | 100 μM (³H) cGMP + cGMP |
| Vehicle: | 1% H₂O |
| Pre-Incubation Time/Temp: | None |
| Incubation Time/Temp: | 20 minutes @ 25° C. |
| Incubation Buffer: | 50 mM Tris-HCl, 5 mM MgCl₂, pH |
| | 7.5 |
| Quantitation Method: | Quantitation of [³H]guanosine |
| Significance Criteria: | ≧50% of max stimulation or |
| | inhibition |

### Reference Compound Data—Biochemical Assays

| | | Reference | Historical | | | Concurrent MIC | |
|---|---|---|---|---|---|---|---|
| Cat. # | Assay Name | Compound | IC₅₀ | Kᵢ | n_{H} | Batch* | IC₅₀ |
| 146000 | Phosphodiesterase | IBMX | 1.1 μM | | | 70431 | 3.76 μM |
| | PDE 1 | | | | | | |
| 148000 | Phosphodiesterase | IBMX | 30 μM | | | 70678 | 81 μM |
| | PDE 2 | | | | | | |
| 152000 | Phosphodiesterase | IBMX | 4.6 μM | | | 70679 | 20.8 μM |
| | PDE 3 | | | | | | |
| 154000 | Phosphodiesterase | IBMX | 6.5 μM | | | 70680 | 30.8 μM |
| | PDE 4 | | | | | | |
| 156000 | Phosphodiesterase | IBMX | 63 μM | | | 70681 | 65.4 μM |
| | PDE 5 | | | | | | |
| 156100 | Phosphodiesterase | Zaprinast | 0.22 μM | | | 70682 | 0.281 μM |
| | PDE 6 | | | | | | |

*Batch: Represents compounds tested concurrently in the same assay(s).

Table 1 is a confidential, private Assay conducted in 2002, the results of which have not been made available to the public.

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

The present invention may be embodied in other specific forms without departing from its spirit or essential characteristics. The described embodiments are to be considered in all respects only as illustrative and not restrictive. The scope of the invention is, therefore, indicated by the appended claims, rather than by the foregoing description. All changes which come within the meaning and range of equivalency of the claims are to be embraced within their scope.

What is claimed and desired to be secured by Letters Patent is:

1. A method comprising:
inhibiting the PDE enzyme in a mammal, said step of inhibiting comprising:
administering to said mammal a composition comprising Morinda citrifolia fruit juice concentrate in an amount sufficient to inhibit the PDE enzyme in the mammal.

2. The method of claim 1, wherein said composition comprises Morinda citrifolia fruit juice obtained by freeze concentrating Morinda citrifolia fruit juice.

3. The method of claim 1, wherein said composition comprises Morinda citrifolia fruit juice concentrate obtained by evaporative concentration of Morinda citrifolia fruit juice.

4. A method for reducing allergy reactions comprising:
administering to a mammal a composition comprising Morinda citrifolia fruit juice concentrate in an amount sufficient to inhibit the action of a PDE enzyme, thereby increasing the level of cAMP, decreasing IgE, and decreasing the secretion of histamine to decrease the allergic reaction.

5. A method for increasing the body energy of a mammal comprising:
administering to a mammal a composition comprising Morinda citrifolia fruit juice concentrate in an amount sufficient to inhibit the action of a PDE enzyme, thereby increasing the level of cAMP, and increasing the amount of glucose produced to increase the body energy of said mammal.

6. A method for reducing asthma reactions comprising:
administering to a mammal a composition comprising Morinda citrifolia fruit juice concentrate in an amount sufficient to inhibit the action of a PDE enzyme, thereby increasing the level of cAMP, and relaxing the muscle around the mammal's airways to decrease the severity of the reaction to asthma.
